(19) United States
(12) Patent Application Publication
(10) Pub. No.: US 2017/0311953 A1
(43) Pub. Date: Nov. 2, 2017

(54) Body Lumen Occlusion Apparatus and Methods

(71) Applicant: Mayo Foundation for Medical Education and Research, Rochester, MN (US)

(72) Inventors: James H. Christensen, Rochester, MN (US); Raymond R. Phelps, Byron, MN (US); Thom W. Rooke, Rochester, MN (US); Henna Kalsi, Rochester, MN (US); Alexander D. Streeter, Rochester, MN (US)

(73) Assignee: Mayo Foundation for Medical Education and Research, Rochester, MN (US)

(21) Appl. No.: 15/629,577

(22) Filed: Jun. 21, 2017

Related U.S. Application Data
(63) Continuation of application No. 13/266,900, filed on Aug. 30, 2012, now abandoned.
(60) Provisional application No. 61/288,007, filed on Dec. 18, 2009.
(60) Provisional application No. 61/174,086, filed on Apr. 30, 2009.

(51) Int. Cl. A61B 17/12; A61B 17/04; A61B 17/00; A61B 17/06

(52) U.S. Cl. CPC A61B 17/12; A61B 17/12172; A61B 17/12163; A61B 17/12036; A61B 17/1214; A61B 2017/0408; A61B 2017/0406; A61B 2017/0404; A61B 2017/0053; A61B 17/12159; A61B 17/12022; A61B 17/12013; A61B 2017/0409; A61B 2017/0417; A61B 17/12009; A61B 2017/061; A61B 2017/0419

(57) ABSTRACT

Body lumen occlusion apparatus and methods are described.

### RELATED APPLICATIONS

This application is a continuation of U.S. patent application Ser. No. 13/266,900 that claims the benefit under 35 U.S.C. §119(e) of U.S. Provisional Patent Application No. 61/174,086, filed on Apr. 30, 2009 and titled BODY LUMEN OCCLUSION APPARATUS AND METHODS and U.S. Provisional Patent Application No. 61/288,007, filed on Dec. 18, 2009 and titled BODY LUMEN OCCLUSION APPARATUS AND METHODS; all of which are hereby incorporated by reference in their respective entireties.

Body lumen occlusion apparatus and methods are described herein.

Occlusion of body lumens within a patient may be used in many different applications, e.g., the closure of blood vessels (e.g., varicose veins, etc.), uterine tubes, seminal vesicles, etc.

### SUMMARY

A variety of different body lumen occlusion apparatus are described herein and can be deployed on and/or within a body lumen to occlude the lumen by compression and/or obstruction. Some of the embodiments described herein include first and second occlusion elements that are connected, while other embodiments include a single occlusion element attached to other structures.

In various embodiments, the occlusion element or elements may be delivered through a delivery device in delivery configuration and, after deployment, take on a deployed configuration. In at least some embodiments, the transition from delivery configuration to the deployed configuration involves a change in size of the occlusion element, e.g., expansion, unfolding, etc. In other embodiments, the transition from delivery configuration to the deployed configuration involves a change in orientation of the occlusion element. In still other embodiments, the transition from delivery configuration to the deployed configuration may involve a change in size and a change in orientation.

The occlusion elements of the various apparatus described herein are preferably deliverable through a delivery device in what may be described as a minimally-invasive procedure. Preferably, the procedure will require only a single point of entry through the skin of a subject, with the delivery device being advanced through two openings made on opposite sides of the lumen to be occluded, followed by deployment of the occlusion elements through a delivery lumen in the delivery device such that the occlusion elements are located on opposite sides of the lumen after the delivery device has been withdrawn.

After the body lumen has been occluded, the occlusion elements may be left in situ where they may dissolve over time. Alternatively, the occlusion elements may be non-dissolvable, in which case the devices are preferably made of biocompatible materials that will, over time, become encased in scar tissue at the site of deployment.

In a first aspect, some embodiments of the body lumen occlusion apparatus described herein may include a first occlusion element; a second occlusion element; a delivery device comprising a first compartment and a second compartment, wherein the first occlusion element is located in the first compartment and the second occlusion element is located in the second compartment; an outer sheath comprising a delivery lumen terminating at a distal end of the outer sheath, wherein the delivery device, the first occlusion element, and the second occlusion element are located within the delivery lumen, wherein the delivery device is movable within the delivery lumen such that the first compartment and the second compartment can be advanced out of the distal end of the outer sheath; and a connector attached to the first occlusion element and the second occlusion element, wherein the connector extends from the first occlusion element to the second occlusion element, wherein the first occlusion element and/or the second occlusion element are movable along the connector such that the first occlusion element and the second occlusion element can be moved towards each other along the connector.

In some embodiments of apparatus according to the first aspect, the apparatus may include ejection structure associated with the first compartment, wherein the ejection structure is configured to selectively force the first occlusion element out of the first compartment when the first compartment and the first occlusion element are outside of the delivery lumen. In some embodiments, the apparatus may also include ejection structure associated with the second compartment, wherein the ejection structure is configured to selectively force the second occlusion element out of the second compartment when the second compartment and the second occlusion element are outside of the delivery lumen.

In some embodiments of the apparatus described herein that include an ejection structure, the ejection structure may comprise an element configured to apply a force to the first occlusion element. The element of the ejection structure may comprise a resilient element and/or an expandable element (e.g., an inflatable element).

In some embodiments of the apparatus described herein that include an ejection structure, the ejection structure may comprise a lumen comprising an orifice located in the first compartment.

In some embodiments of apparatus according to the first aspect, the apparatus may include the distal end of the outer sheath comprises tissue piercing structure.

In some embodiments of apparatus according to the first aspect, the connector extends proximally from the second occlusion element to a proximal end of the delivery device.

In some embodiments of apparatus according to the first aspect, the connector comprises suture material threaded through an opening in the first occlusion element and/or the second occlusion element. In some embodiments, the connector comprises two lengths of suture material extending between the first occlusion element and the second occlusion element, and further wherein the delivery device comprises first and second channels extending between the first compartment and the second compartment, wherein one length of the suture material is located in the first channel and the other length of suture material is located in the second channel.

In some embodiments of apparatus according to the first aspect, the apparatus may include an intraluminal obstruction element is attached to the connector and located between the first occlusion element and the second occlusion element. In some embodiments, the intraluminal obstruction element comprises an embolization coil.

In a second aspect, a body lumen occlusion apparatus as described herein may include a delivery device having a delivery lumen, wherein the delivery lumen opens at a distal port that is located at a distal end of the delivery device, and wherein the distal end of the delivery device optionally includes tissue piercing structure; a first occlusion element having a delivery configuration in which the first occlusion element can be advanced through the delivery lumen and exit from the delivery lumen of the delivery device through the distal port, wherein the first occlusion element further includes a deployed configuration after the first occlusion element is advanced out of the distal port of the delivery lumen, and wherein the delivery lumen restrains the first occlusion element in the delivery configuration when the first occlusion element is located within the delivery lumen; a second occlusion element having a delivery configuration in which the second occlusion element can be advanced through the delivery lumen and exit from the delivery lumen of the delivery device through the distal port, wherein the second occlusion element further includes a deployed configuration after the second occlusion element is advanced out of the distal port of the delivery lumen, and wherein the delivery lumen restrains the second occlusion element in the delivery configuration when the second occlusion element is located within the delivery lumen; and a connector attached to the first occlusion element and the second occlusion element, wherein the connector extends from the first occlusion element to the second occlusion element through the delivery lumen of the delivery device, wherein the second occlusion element is movable along the connector within the delivery lumen such that the second occlusion element can be moved towards the first occlusion element along the connector; and wherein the connector extends proximally from the second occlusion connector to a proximal end of the delivery device.

In some embodiments, apparatus according to the second aspect described herein the connector may include suture material threaded through an opening in the second occlusion element. In still other embodiments, the suture material may be threaded through the first occlusion element. In still other embodiments, the connector may be in the form of a single, continuous length of the suture material, wherein the suture material is threaded through two openings in the first occlusion element and two openings in the second occlusion element. In still other embodiments, the suture material may include two free ends extending out of the proximal end of the delivery device. In still other embodiments, the apparatus may include a cartridge containing the first occlusion element and a portion of the connector, wherein the cartridge mates with the proximal end of the delivery device.

In some embodiments of apparatus according to the second aspect, the delivery configuration of the first occlusion element may include one or more folds that are expanded or unfolded to transition the first occlusion element to the deployed configuration when the first occlusion element is advanced out of the distal port of the delivery lumen. In still other embodiments, the delivery configuration of the second occlusion element may also include one or more folds that are expanded or unfolded to transition the second occlusion element to the deployed configuration when the second occlusion element is advanced out of the distal port of the delivery lumen.

In some embodiments of apparatus according to the second aspect, the first occlusion element may include a generally disc-shaped element, and the delivery configuration of the first occlusion element may involve canting the first occlusion element within the delivery lumen. In still other embodiments, the second occlusion element of such an apparatus may also be a generally disc-shaped element, and the delivery configuration of the second occlusion element may involve canting the second occlusion element within the delivery lumen.

In some embodiments of apparatus according to the second aspect, the apparatus may include a stylet sized to advance through the delivery lumen, wherein the stylet has a passageway through which the connector passes to the proximal end of the delivery lumen. In still other embodiments, the passageway may be in the form of a channel formed in an outer surface of the stylet. In still other embodiments, the stylet may include a flange positioned proximate a proximal end of the stylet, wherein the flange limits distal advancement of the stylet through the delivery lumen.

In some embodiments of apparatus according to the second aspect, the apparatus may include an intraluminal obstruction element is attached to the connector and located between the first occlusion element and the second occlusion element. In some embodiments, the intraluminal obstruction element comprises an embolization coil.

In a third aspect, a body lumen occlusion apparatus as described herein may include a delivery device comprising a delivery lumen, wherein the delivery lumen opens at a distal port that is located at a distal end of the delivery device, and wherein the distal end of the delivery device comprises tissue piercing structure; a helical anchor sized for advancement through the delivery lumen; an occlusion element comprising a delivery configuration in which the occlusion element can be advanced through the delivery lumen and exit from the delivery lumen of the delivery device through the distal port, wherein the occlusion element further comprises a deployed configuration after the occlusion element is advanced out of the distal port of the delivery lumen, and wherein the delivery lumen restrains the occlusion element in the delivery configuration when the occlusion element is located within the delivery lumen; and a connector attached to the helical anchor and the occlusion element, wherein the connector extends from the helical anchor to the occlusion element through the delivery lumen of the delivery device, wherein the occlusion element is movable along the connector such that the occlusion element can be moved towards the helical anchor along the connector.

In some embodiments of the third aspect of apparatus as described herein, the connector extends proximally from the occlusion element to a proximal end of the delivery device.

In some embodiments of the third aspect of apparatus as described herein, the occlusion element comprises a support structure and a flexible sheet attached to the support structure.

In some embodiments of the third aspect of apparatus as described herein, an intraluminal obstruction element is attached to the connector and located between the occlusion element and the helical anchor. In some embodiments, the intraluminal obstruction element comprises an embolization coil.

In another aspect, some embodiments of methods of using some of the apparatus described herein to occlude a body lumen may include advancing the delivery device to a selected location, wherein the advancing comprises piercing two opposing sides of a body lumen; deploying the first occlusion element on a distal side of the body lumen; deploying the second occlusion element on the proximal side of the body lumen; and drawing the first occlusion element and the second occlusion element together, wherein the body lumen is occluded between the first occlusion element and the second occlusion element, and wherein the first occlusion element and the second occlusion element are connected to each other through the body lumen by the connector.

In another aspect, some embodiments of methods of using some of the apparatus described herein to occlude a body lumen may include advancing the delivery device to a selected location, wherein the advancing comprises piercing at least one side of a body lumen; attaching the helical anchor to one side of the body lumen; deploying the occlusion element on an opposing side of the body lumen; and drawing the occlusion element and the helical anchor together along the connector, wherein the body lumen is occluded between the occlusion element and the helical anchor, and wherein the occlusion element and the helical anchor are connected to each other through the body lumen by the connector.

In another aspect, some embodiments of methods of using some of the apparatus described herein to occlude a body lumen may include advancing the delivery device to a selected location, wherein the advancing involves piercing two opposing sides of a body lumen; deploying the first occlusion element on a distal side of the body lumen; withdrawing the delivery device to a proximal side of the body lumen; deploying the second occlusion element on the proximal side of the body lumen; and drawing the first occlusion element and the second occlusion element together, wherein the body lumen is occluded between the first occlusion element and the second occlusion element, and wherein the first occlusion element and the second occlusion element are connected to each other through the body lumen by the connector.

The words “preferred” and “preferably” as used herein refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein and in the appended claims, “a,” “an,” “the,” “at least one,” and “one or more” are interchangeable unless explicitly limited to the singular form or situations in which the context clearly dictates otherwise.

The term “and/or” means one or all of the listed elements or a combination of any two or more of the listed elements.

The above summary is not intended to describe each embodiment or every implementation of the present invention. Rather, a more complete understanding of the invention will become apparent and appreciated by reference to the following Description of Illustrative Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1 is a side view of one embodiment of an occlusion element as described herein;

FIG. 2 is a side view of the occlusion element of FIG. 1 in what may be described as a delivery configuration;

FIG. 3 is a partial cross-sectional view depicting the occlusion element of FIGS. 1 and 2 within the delivery lumen of a delivery device;

FIG. 4 is a side view of another embodiment of an occlusion element as described herein;

FIG. 5 is a plan view of the occlusion element of FIG. 4;

FIG. 6 is a partial cross-sectional view depicting an occlusion element located within the delivery lumen of a delivery device, and wherein the delivery device has been advanced through the skin and two opposing sides of a body lumen;

FIG. 7 is a partial cross-sectional view depicting the occlusion element of FIG. 6 after deployment on a distal side of the body lumen;

FIG. 8 is a partial cross-sectional view depicting a second occlusion element located within the delivery lumen of a delivery device after the first occlusion element has been deployed as depicted in FIG. 7;

FIG. 9 is a partial cross-sectional view of the occlusion apparatus of FIG. 8 after deployment of the second occlusion element from the delivery device on a proximal side of the body lumen;

FIG. 10 is a partial cross-sectional view of the occlusion apparatus of FIG. 9 depicting compression of the body lumen between the deployed occlusion elements;

FIG. 11 is a side view of another embodiment of an occlusion element;

FIG. 12 is a partial cross-sectional view of the occlusion element of FIG. 11 in a delivery lumen of a delivery device;

FIG. 13 is a partial cross-sectional view of an occlusion apparatus during deployment through a body lumen;

FIG. 14 is a partial cross-sectional view of the occlusion apparatus of FIG. 13 deployed on opposing sides of a body lumen;

FIG. 15 is a partial cross-sectional view of the occlusion apparatus of FIG. 14 depicting compression of the body lumen between the deployed occlusion elements;

FIG. 16 is a side view of one embodiment of a delivery device that may be used in the occlusion apparatus described herein;

FIG. 17 is an enlarged perspective view of a distal end of the delivery device of FIG. 16;

FIG. 18 is a side section view of the portion of the delivery device depicted in FIG. 17;

FIG. 19 is a side view of one embodiment of a pusher that may be used to advance occlusion elements through a delivery device of the occlusion apparatus described herein;

FIG. 20 is an enlarged view of the distal end of the pusher of FIG. 19;

FIGS. 21-23 depict various views a pair of occlusion elements and an associated connector that may be used in the occlusion apparatus described herein;

FIG. 24 is a cross-sectional view of a cartridge assembly that may be used to load an occlusion element in a delivery device of the occlusion apparatus described herein; and

FIG. 25 is a side view of another embodiment of an occlusion apparatus in which the occlusion elements are connected by a mechanical connector.

FIG. 26 depicts another embodiment of an occlusion apparatus.

FIGS. 27A-27C depict various embodiments of occlusion elements that could potentially be used with the occlusion apparatus of FIG. 26.

FIGS. 28A & 28B depict another embodiment of an occlusion apparatus before delivery and after deployment.

FIGS. 29-34 depict a variety of embodiments of occlusion apparatus including an intraluminal obstruction element.

FIG. 35 depicts another embodiment of an occlusion apparatus in form of a coil spring.

FIGS. 36 & 37 depict additional embodiments of occlusion apparatus for occluding a lumen.

FIGS. 38A-38D depict another embodiment of an occlusion apparatus including a delivery device having occlusion element compartments therein.

FIGS. 39A-39B depict another embodiment of an occlusion apparatus including a delivery device using a pushing element to deliver occlusion elements.

FIGS. 40A-40D depict another embodiment of an occlusion apparatus including a delivery device having occlusion element compartments therein.

FIGS. 41A-41B depict occlusion elements with an optional guide to address potential tissue tearing.

### DESCRIPTION OF ILLUSTRATIVE EMBODIMENTS

In the following description of illustrative embodiments, reference is made to the accompanying figures of the drawing which form a part hereof, and in which are shown, by way of illustration, specific embodiments that may be practiced. It is to be understood that other embodiments may be utilized and structural changes may be made without departing from the scope of the present invention.

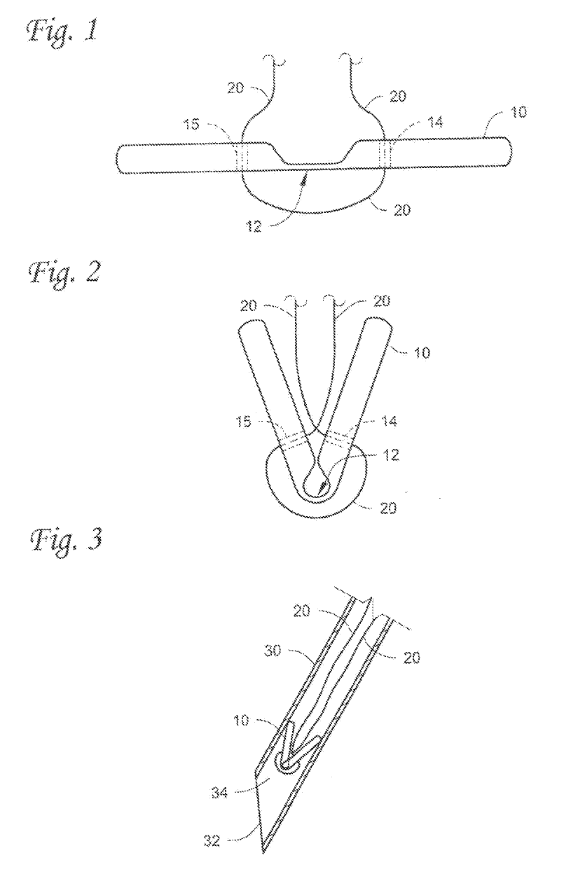

FIGS. 1 and 2 depict one embodiment of an occlusion element that may be used in occlusion apparatus as described herein. The occlusion element 10 is in the form of an elongated body, e.g., rod-shaped and includes a thinned section 12 that is adapted to allow the device 10 to fold upon itself as depicted in FIG. 2.

Also depicted in FIGS. 1 and 2 is a connector 20 that is threaded through openings 14 and 15 in the device 10. The connector 20 may be in the form of suture material. The suture material may or may not be constructed of dissolvable (e.g., bio-resorbable) materials. The connector 20 may loop around the folded portion of the folded occlusion element 10 as seen in FIG. 2.

Although the connector 20 is depicted in the form of a continuous length of material, in some embodiments, the connector 20 may be provided as two separate and distinct elements that are separately attached to the occlusion element 10 (using, e.g., knots, adhesives, mechanical fasteners, etc.). In still other embodiments, the connector 20 may be provided in the form of a single element that is attached to the occlusion element 10, with only a single element extending proximally through a delivery device as described herein.

FIG. 3 is a partial cross-sectional view depicting the occlusion element 10 of FIGS. 1 and 2 within the delivery lumen 34 of a delivery device 30. The delivery device 30 may be in the form of, e.g., a needle or other elongate device that can be advanced through tissue to a selected internal body location. It may be preferred that the delivery device 30 include a distal end 32 that includes tissue piercing structure (such as, e.g., a narrowed tip, sharpened edges, etc.).

The occlusion element 10 is depicted in FIG. 3 in what may be characterized as its delivery configuration which, for the occlusion element 10, is similar to the folded configuration depicted in FIG. 2. The connector 20 is also depicted in FIG. 3 as extending proximally from the occlusion element 10 through the delivery lumen 34 of the delivery device. Although the occlusion element 10 is depicted as being advanced with its folded end leading, the occlusion element could alternatively be advanced through the lumen 34 in another orientation.

Referring to FIGS. 1-3, the occlusion element 10 can be described as having a delivery configuration which, for the depicted embodiment is folded as described above, and a deployed configuration as seen in, e.g., FIG. 1 in which the occlusion element 10 is unfolded or generally flat. The occlusion element 10 is typically free to move from its delivery configuration to the deployed configuration after it is advanced out the distal port of the delivery lumen 34 in the delivery device 10 as is described herein.

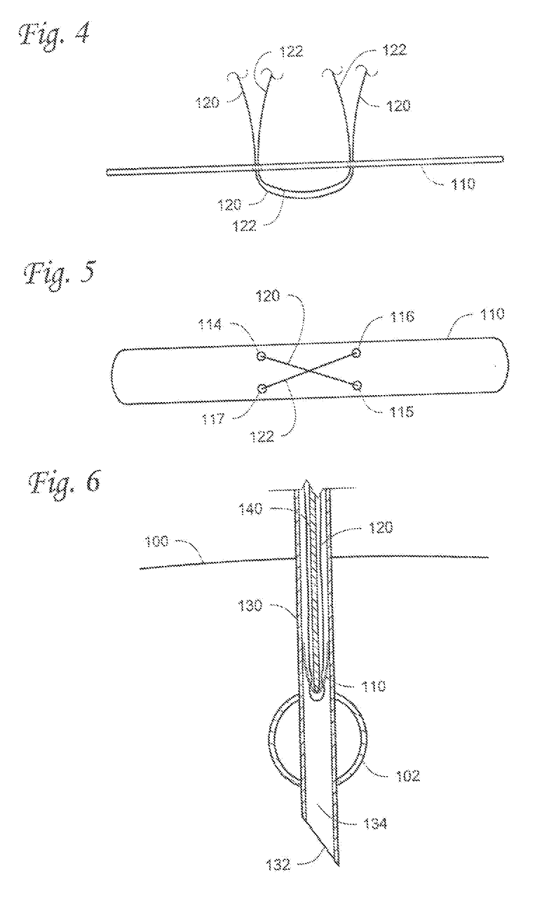

FIGS. 4 and 5 depict an alternative embodiment of an occlusion element 110 that is in the form of a generally flat strip as opposed to the more rod-like shape of the occlusion element 10 of FIGS. 1-3. Another optional feature depicted in FIGS. 4-5 is the use of two connectors 120 and 122. The connector 120 is threaded through openings 114 and 115 in the occlusion element 110. The connector 122 is threaded through openings 116 and 117 in the occlusion element.

FIG. 6 is a partial cross-sectional view of an occlusion apparatus including the occlusion element 110 of FIGS. 4-5 located within the delivery lumen 134 of a delivery device 130. The delivery device 130 is depicted in FIG. 6 as having been advanced through the skin 100 and two opposing sides of a body lumen 102 such that distal tip 132 is located past the distal side of the body lumen 102. Also seen in FIG. 6 is a pusher 140 that is being used to advance the occlusion element 110 distally through the delivery lumen 132 of the delivery device 130. The occlusion element 110 is in a delivery configuration (which is folded in this embodiment) such that it can be advanced through the delivery lumen 134.

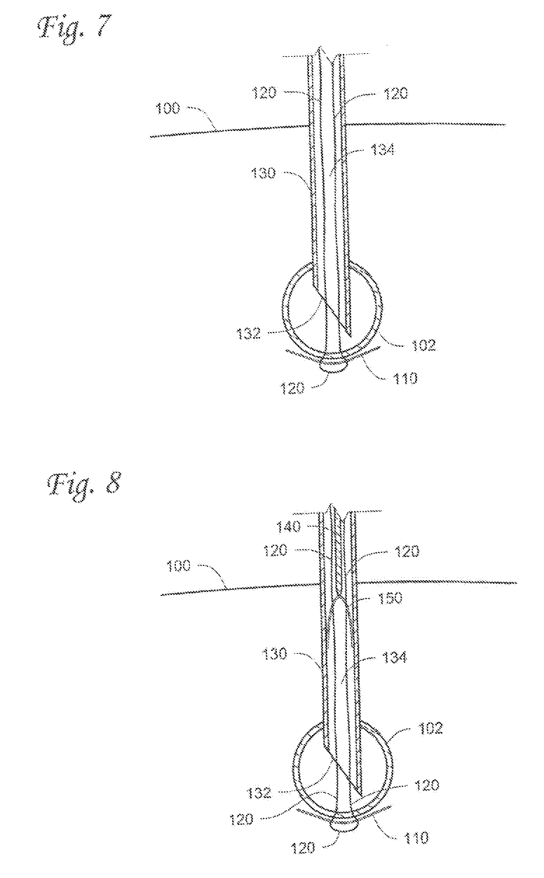

FIG. 7 depicts the occlusion apparatus occlusion of FIG. 6 after deployment of the occlusion element 110 on a distal side of the body lumen 102. The occlusion element 110 is depicted in FIG. 7 in its deployed configuration (which, in the depicted embodiment, is unfolded from the folded delivery configuration). The pusher 140 has also been withdrawn in the proximal direction out of the delivery lumen 134 as depicted in FIG. 7.

FIG. 8 depicts a second occlusion element 150 located within the delivery lumen 134 of the delivery device 130 after the first occlusion element 110 has been deployed. Also seen in FIG. 8 is the distal end of the pusher 140 (which may be the same pusher as depicted in FIG. 6 or a different one). The pusher is used to advance the second occlusion element 150 in the distal direction through the delivery lumen 134. The second occlusion element 150 preferably advances distally over the connector 120 which may preferably remain substantially fixed or stationary within the lumen 134 (e.g., the connector 120 is neither being advanced or retracted).

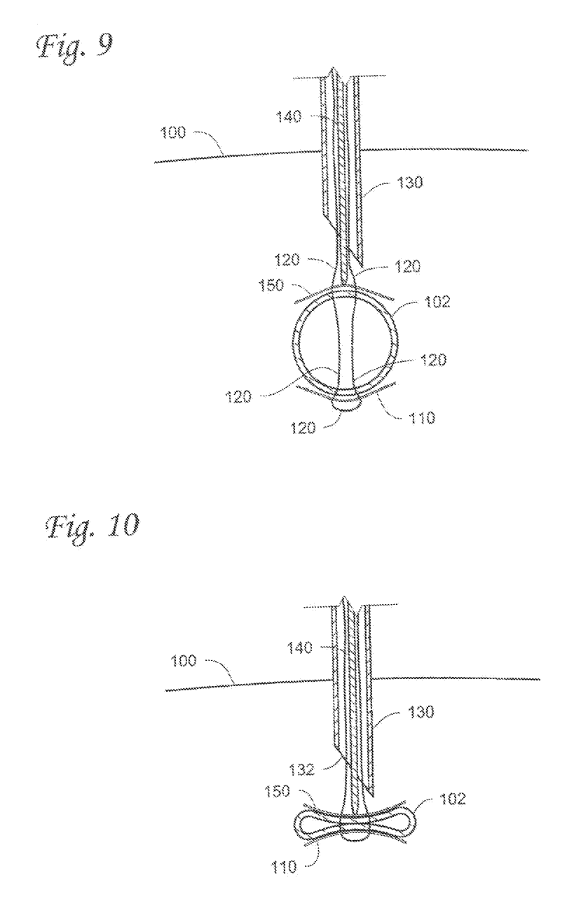

FIG. 9 depicts the occlusion apparatus of FIG. 8 after deployment of the second occlusion element 150 from the delivery device 130 on a proximal side of the body lumen 102. The occlusion element 150 has transitioned from its delivery configuration (as seen in FIG. 8) to its deployed configuration. The delivery device 130 is also depicted as having been withdrawn proximally from the body lumen 102 such that the delivery device 130 is no longer piercing or located within the lumen 102.

FIG. 10 depicts the occlusion apparatus of FIG. 9 after compression of the body lumen 102 between the deployed occlusion elements 110 and 150. In some instances the compression may be obtained by providing tension using the connector 120 while preventing proximal movement of the occlusion element 150 using the distal end 132 of the delivery device 130. Alternatively or in addition to these techniques, the pusher 140 may also be used to obtain the desired compression of the body lumen 102.

The tension in the connector 120 and the corresponding compression of the body lumen 102 may be maintained by any suitable technique or combination of techniques. For example, the connector 120, if in the form of suture material, may be tied, a suture lock may be advanced over the suture material, adhesive may be applied to the connector, the connector 120 may be bonded to itself and/or one or both of the occlusion element 110, 150, etc.

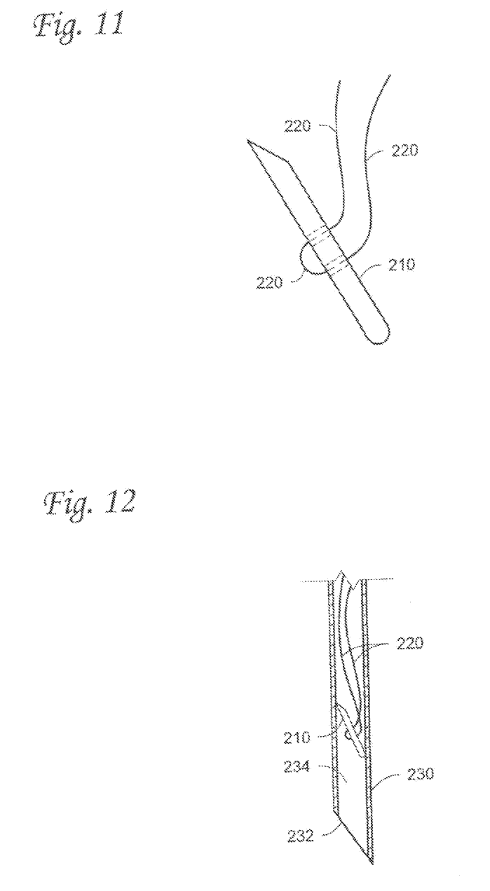

FIG. 11 depicts another embodiment of an occlusion element 210 that may be used in occlusion apparatus described herein, while FIG. 12 depicts the occlusion element 210 of FIG. 11 in a delivery lumen 234 of a delivery device 230, with a connector 220 extending proximally through the delivery lumen 234 from the occlusion element 210. The delivery device 230 also preferably includes a distal end 232 in the form of a tissue-piercing structure.

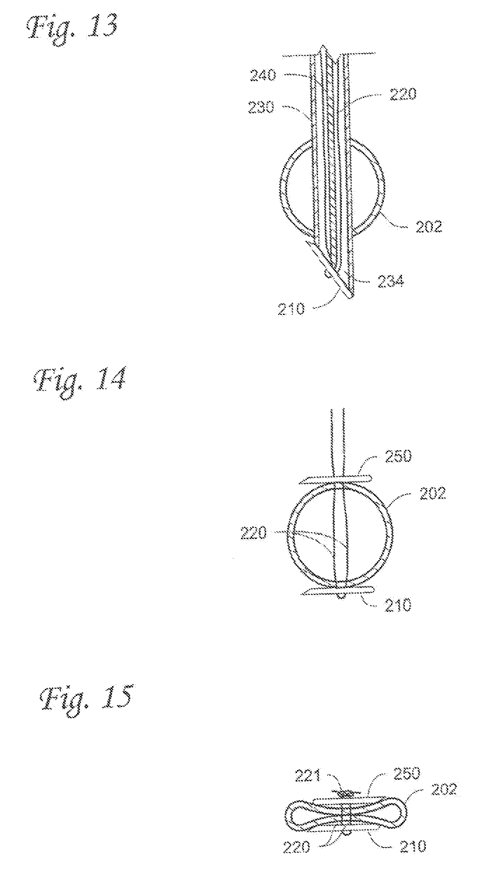

FIGS. 13-14 depict steps in the process of deploying the occlusion element 210 and its opposing element 250 on opposite sides of a body lumen 202. The process involves the use of a pusher 240 as discussed herein to advance the occlusion elements through the delivery lumen 234 of the delivery device 230. FIG. 15 depicts the occlusion apparatus of FIGS. 11-14 after compression of the body lumen 202 between the deployed occlusion elements 210 and 250, with the compression being maintained by a knot 221 in the embodiment depicted in FIG. 15 (although a variety of other techniques could be used in place of or in addition to the knot 221 as discussed herein).

One difference between the occlusion elements 210 and 250 depicted in FIGS. 11-15 from those previously described is that the occlusion elements 10, 110, and 150 all involve expansion (e.g., unfolding) when transitioning from their delivery configurations (where they include at least one fold) to their deployed configurations. In contrast, the occlusion elements 210 and 250 are in the form of generally disc-shaped elements. The occlusion elements 210 and 250 are not, however, limited to circular disks (e.g., the may be hexagonal, octagonal, oval, some irregular shape, etc.) and they may include some variations in thickness between the major faces. The occlusion elements 210 and 250 transition from a canted orientation within the delivery lumen 234 to a deployed configuration in which the occlusion elements 210 and 250 are generally parallel to each other and are generally transverse to an axis extending through the relatively straight delivery lumen 234. The delivery lumen 234 may have a profile that does not match the shape of the occlusion elements 210 and 250 such that, upon deployment of the occlusion elements 210 and 250, they cannot typically re-enter the delivery lumen 234 unless they are in a canted orientation (e.g., as delivered). If, e.g., the occlusion elements 210 and 250 are generally circular disks, the delivery lumen 234 may have a non-circular profile (e.g., a profile that is in the form of an oval, ellipse, rectangle, etc.).

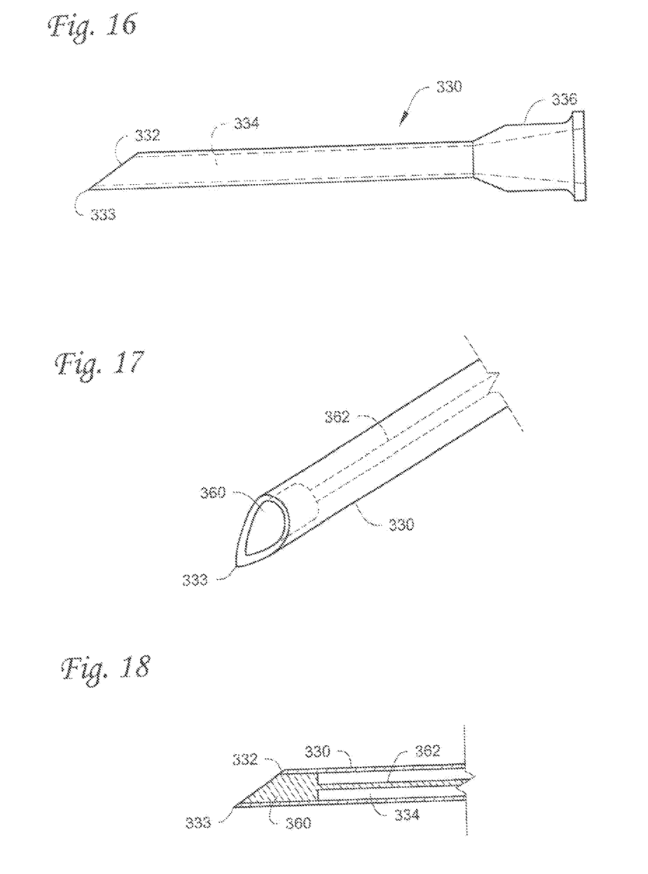

FIGS. 16-18 depict various features of one embodiment of a delivery device 330 that may be used in connection with the occlusion apparatus described herein. The delivery device 330 includes a delivery lumen 334 that extends from the proximal end of the delivery device (which includes handle 336) to the distal end 332. The delivery device 330 may preferably include a tissue piercing tip 333 as seen in FIGS. 16-18. Although not required, the delivery device 330 may be in the form a relatively rigid needle.

Another feature depicted primarily in FIGS. 17-18 is a stylet 360 that may be positioned at the distal end 332 of the delivery device 330 to reduce the likelihood of tissue coring as the delivery device 330 is advanced through tissue. The stylet 360 may include a proximally extending element 362 that can be used to remove the stylet 360 before advancing occlusion element through the delivery lumen 334 as described herein.

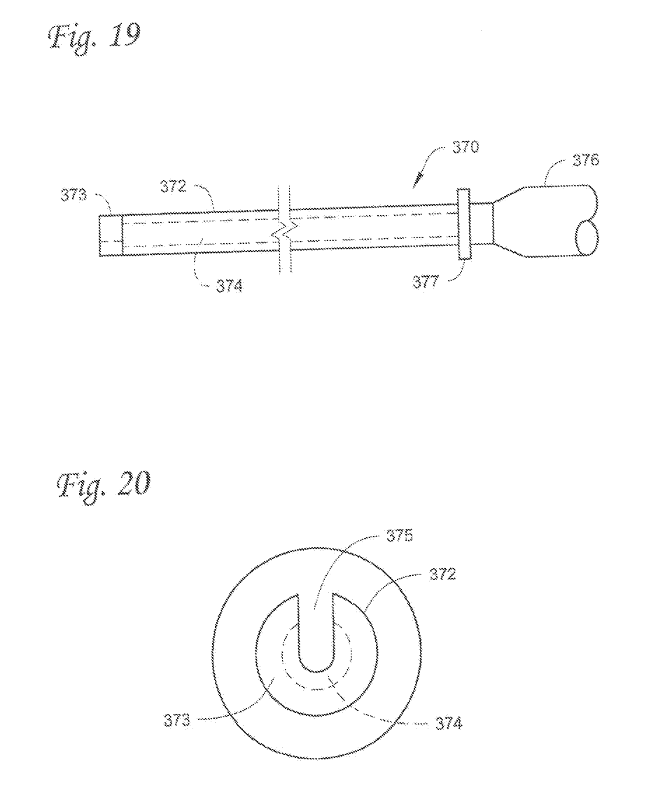

FIG. 19 depicts one embodiment of a pusher 370 that may be used to advance occlusion elements through a delivery device of the occlusion apparatus as described herein. FIG. 20 is an enlarged view of the distal end of the pusher of FIG. 19. The pusher 370 of FIGS. 19-20 includes an elongate body 372 that includes a channel 374 formed therein. The channel 374 may be used to allow the connectors to pass therethrough while the pusher is being used to advance occlusion elements through a delivery lumen. The distal end 373 of the pusher 370 may also include a slot 375 that facilitates passage of the connectors attached to the occlusion elements into the channel 374 of the pusher 370.

The pusher 370 may include a handle 376 and a flange 377 that is preferably sized to limit the distance to which the distal end 373 of the pusher 370 can be advanced through a delivery lumen. The proximal end of the delivery device 330 may include, for example, a Luer Lock fitting for the attachment of other components or devices thereto.

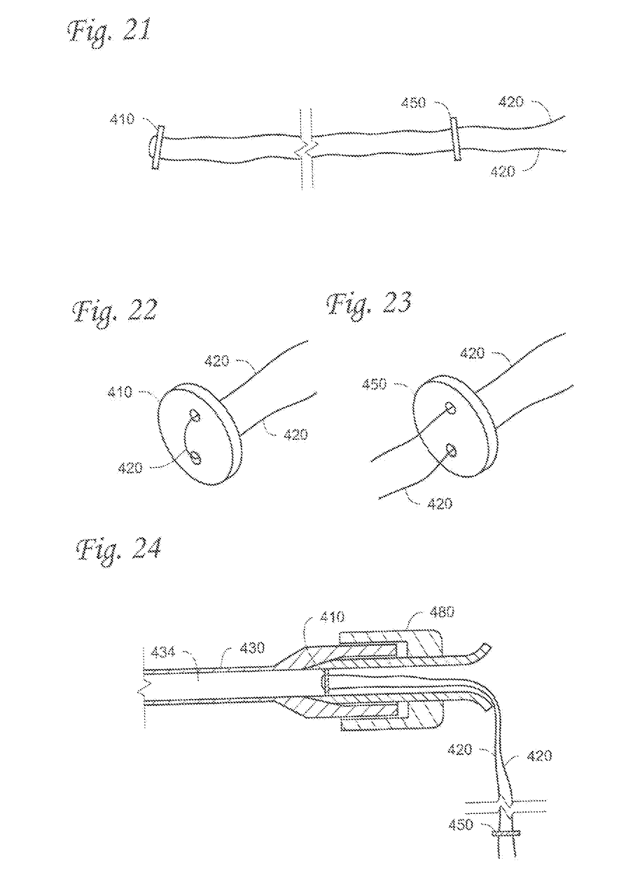

FIGS. 21-23 depict various views a pair of occlusion elements 410 and 450 and an associated connector 420 that may be used in an occlusion apparatus described herein. Such an assembly of occlusion elements 210 and 250 along with connector 220 may be conveniently delivered to and loaded into a delivery device using a cartridge 480 as depicted in FIG. 24.

The cartridge 480 may be adapted to mate with the proximal end of the delivery device 430 such that the first occlusion element 410 is positioned for insertion into and advancement through the delivery lumen 434 of the delivery device 430. If, e.g., the proximal end of the delivery device 430 includes a Luer lock, then the cartridge 480 may be adapted for use with a Luer lock. Further, the second occlusion element 450 may be conveniently positioned for advancement into the delivery lumen 434 after the first occlusion element 410 is deployed as described herein.

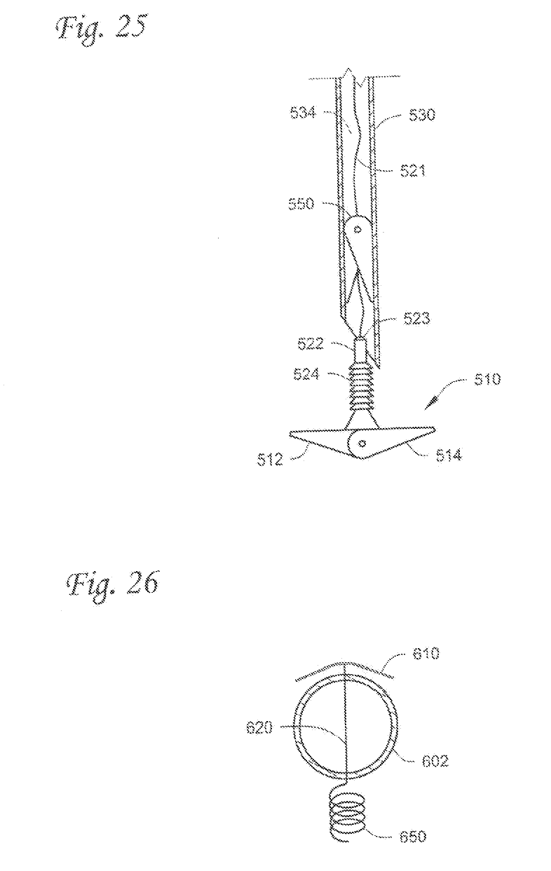

FIG. 25 is a side view of another embodiment of an occlusion apparatus in which the occlusion elements 510 and 550 are connected by a mechanical connector after deployment. The first occlusion element 510 is depicted in a deployed configuration in which the arms 512 and 514 are opened. The second occlusion element 550 is depicted as still remaining in its delivery configuration (e.g., folded) in the delivery lumen 534 of the delivery device 530.

The connector used to connect the occlusion element 510 and 550 to occlude a body lumen as described herein may include a flexible element 521 that is attached to the proximal end 523 of a post 522 that is attached to the occlusion element 510. The post 522 may preferably include a series of serrations, grooves or other structures 524 that cooperate with complementary ratcheting structure on the other occlusion element 550 to connect the occlusion elements 510 and 550 with a body lumen being compressed between them.

The flexible element 521 of the connector may preferably extend out of the proximal end of the delivery device 530 where is could be used to apply forces that can, in turn advance the occlusion elements towards each other to compress a body lumen. In at least some respects, the connector structure of this embodiment may operate on principles similar to those used in conventional toggle bolts and ratcheting wire connectors (sometimes referred to as “zip ties” or wire ties).

FIG. 26 depicts another embodiment of an occlusion apparatus that includes an occlusion element 610 and a helical anchor 650 connected to each other through a lumen 602. The helical anchor 650 may be delivered through a lumen in a delivery device in a straightened or other compressed configuration, with the helical anchor expanding after it is deployed (as depicted in FIG. 26). The occlusion element 610 is connected to the helical anchor 650 through a connector 620 attached to the helical anchor 650 and the occlusion element 610. The connector 620 extends from the helical anchor 650 to the occlusion element 610 through the delivery lumen of a delivery device at the beginning of the delivery process. When deployed to occlude a lumen 602, the occlusion element 610 is movable along the connector 620 such that the occlusion element 610 can be used to occlude a lumen by compression between the helical anchor 650 and the occlusion element 610.

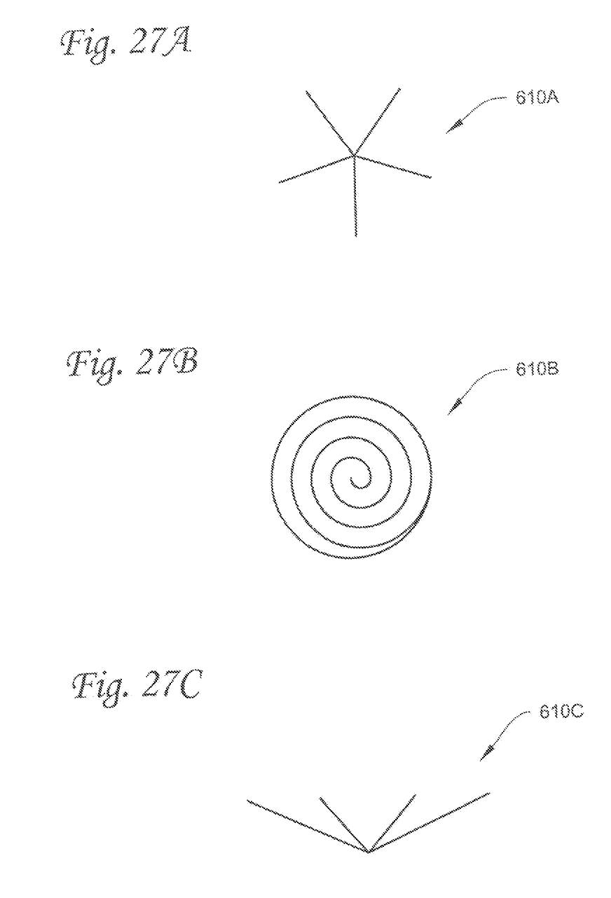

The occlusion element 610 may take a variety of different forms and may, in some embodiments, be collapsible such that the occlusion element 610 can be delivered using a smaller delivery lumen in a delivery device. FIGS. 27A-27C depict some potentially useful configurations for the collapsible occlusion element 610. FIG. 27A depicts a collapsible occlusion element 610A in the form of a star-shaped device, FIG. 27B depicts a collapsible occlusion element 610B in the form of a coil and FIG. 27C depicts a collapsible occlusion element 610C in the form of a umbrella-like shape. In some embodiments, the collapsible occlusion elements may be formed of a wire (made of, e.g., metals, polymers, etc.) while in other embodiments, the collapsible occlusion elements may include a film, fabric, etc. attached to an underlying support (such as, e.g., the structures depicted in FIGS. 27A-27C.

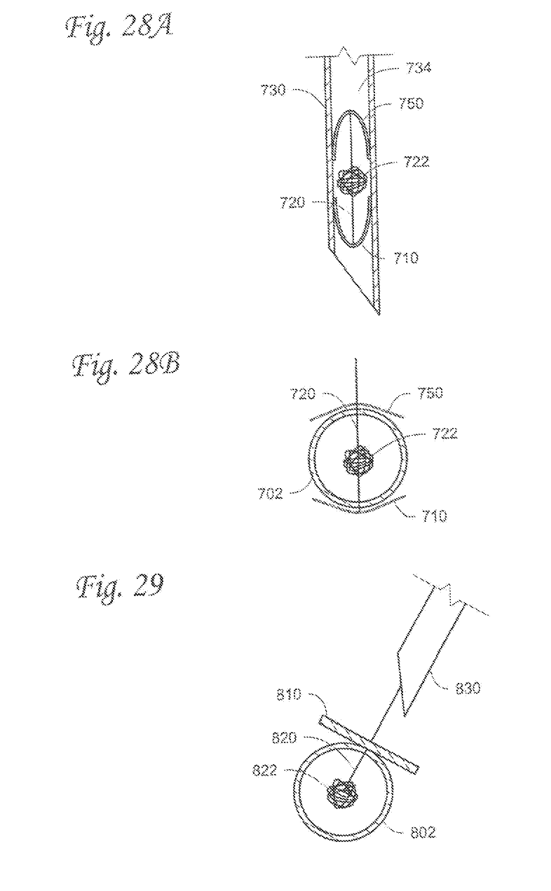

In some embodiments, the occlusion apparatus described herein may include an intraluminal obstruction element provided within the lumen to enhance closure or obstruction of the lumen. One example of such an element is depicted in connection with FIGS. 28A and 28B in which a pair of occlusion elements 710 and 750 are connected to each other by a connector 720. The apparatus includes, in addition to the other elements described in connection with the apparatus described herein, an intraluminal obstruction element 722 that is preferably connected to the connector 720 such that, upon deployment of the apparatus to obstruct a lumen, the intraluminal obstruction element 722 is located within the lumen.

FIG. 28A depicts the apparatus in the delivery lumen 734 of a delivery device 730 while FIG. 28B depicts the apparatus after deployment, in which the intraluminal obstruction element 722 is positioned in the lumen 702 while the occlusion elements 710 and 750 are positioned on opposite sides of the lumen 702 and connected to each other and the intraluminal obstruction element 722 by a connector 720. The occlusion elements 710 and 750 may be advanced towards each other to assist in occluding the lumen 702 through compression, although in some embodiments, such compression may not be required or may be required to a limited degree in view of the intraluminal obstruction element 722 positioned within the lumen 702.

The intraluminal obstruction element 722 may have a variety of different constructions to assist in obstruction of lumen. Some potentially suitable constructions may include, for example, a mesh, fabric, coil (e.g., embolization coil, etc.), collagen plug, adhesive, etc. In some embodiments, the obstruction element may also include thrombin and/or a thrombin coating to assist obstruction of a lumen.

Another embodiment of an occlusion apparatus as described herein is depicted in FIG. 29 and includes an occlusion element 810 designed or adapted for extraluminal placement outside of a lumen 802 and an intraluminal obstruction element 822 connected to the occlusion element 810 by a connector 820. The obstruction element 822 may be delivered into the lumen 802 using a delivery device 830 that preferably pierces a wall of the lumen 802 to allow deployment of the obstruction element 822 in the lumen 802 and positioning of the occlusion element 810 on the outside of the lumen 802. The obstruction element 822 may take any of the forms as described above in connection with obstruction element 722.

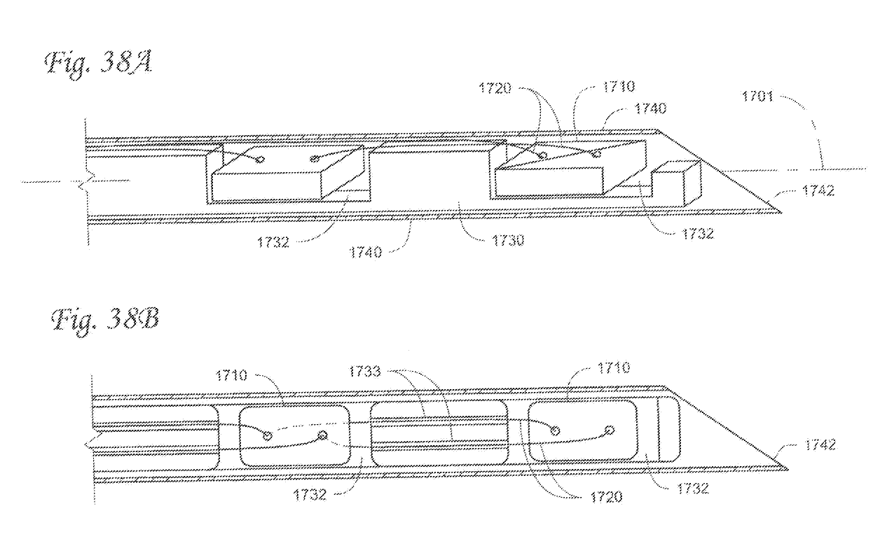

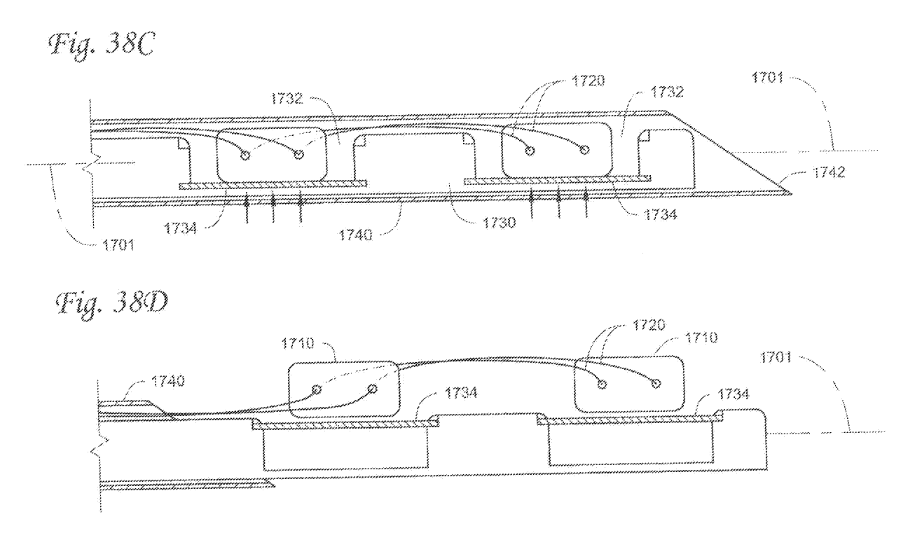

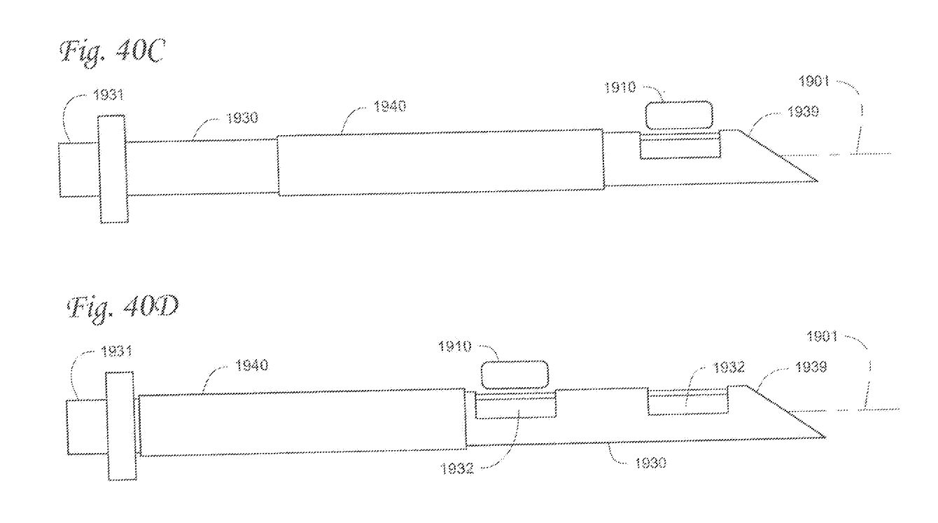

Other embodiments of the occlusion apparatus described herein may also include an intraluminal obstruction element in connection with the one or two occlusion elements even though not depicted or explicitly described in connection with those embodiments. In those embodiments that, for example, include first and second occlusion elements attached to each other by a connector as described herein, an intraluminal obstruction apparatus may be attached to the occlusion elements by the connector and located between the first and second occlusion elements such that it can be positioned at a location within the body lumen to be occluded. Where, for example, the occlusion elements are deployed from a delivery device including compartments (see, e.g., the illustrative embodiments depicted in FIGS. 38A-38D and 40A-40D), the delivery device may include a compartment in which the intraluminal obstruction element is positioned when the in the outer sheath (before deployment). In those embodiments that, for example, include an occlusion element and a helical anchor attached to each other by a connector, an intraluminal obstruction apparatus may be attached to the occlusion element and the anchor by the connector and located between the occlusion element and the anchor such that it can be positioned at a location within the body lumen to be occluded.

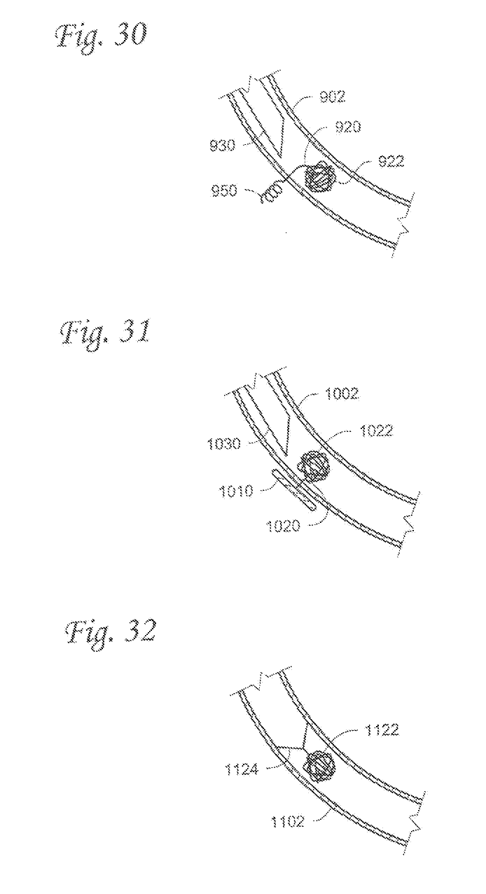

Still another embodiment of an occlusion apparatus as described herein is depicted in FIG. 30 in which an obstruction element 922 is deployed within a lumen 902 with an anchor 950 in the form of a helix piercing the wall of the lumen 902 and connected to the obstruction element 922 by a connector 920. The helical anchor 950 may preferably be capable of piercing the wall of the lumen 902 when deployed from the interior of the lumen such that the occlusion apparatus can be delivered to a selected location within the lumen 902 by advancing a delivery device 930 through the lumen 902. The delivery device 930 may be in the form of, e.g., a catheter, etc. As part of the deployment process, the helical anchor 950 preferably pierces the wall of the lumen 902 to assist in holding the obstruction element 922 in position in the lumen 902. The obstruction element 922 may take any of the forms as described above in connection with obstruction element 722.

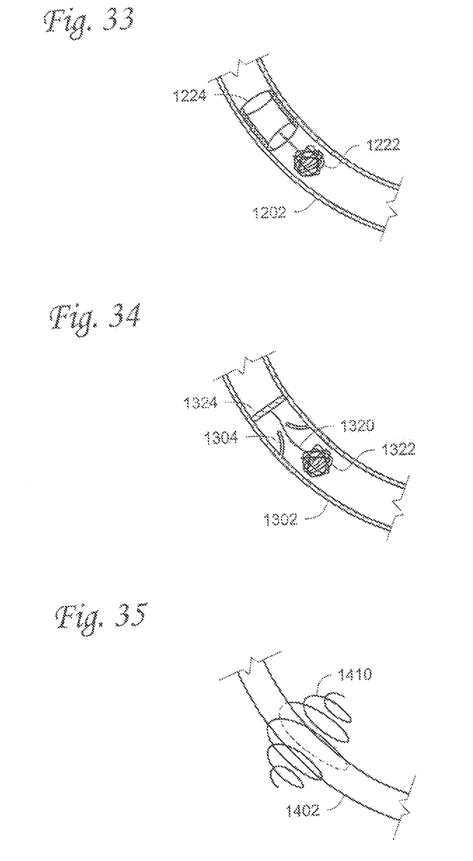

FIG. 31 depicts another embodiment of an occlusion apparatus in which a collapsible occlusion element 1010 is used in place of the helical anchor 950 of the occlusion apparatus depicted in FIG. 30. The occlusion apparatus may preferably be delivered to a selected location in the lumen 1002 using a delivery device 1030 that is advanced through the lumen 1002. The occlusion apparatus includes an obstruction element 1022 that is deployed within a lumen 1002 with a collapsible occlusion element 1010 connected to the obstruction element 1022 by a connector 1020. The collapsible occlusion element 1010 may take a variety of forms as discussed, e.g., in connection with FIGS. 27A-27C. Like the helical anchor of FIG. 30, the collapsible occlusion element 1010 may preferably be capable of piercing the wall of the lumen 1002 during the deployment process. The obstruction element 1022 may take any of the fauns as described above in connection with obstruction element 722.

Still another embodiment of an occlusion apparatus as described herein is depicted in FIG. 32. The occlusion apparatus depicted in FIG. 32 includes an intraluminal anchor 1124 to which an obstruction element 1122 is attached. When deployed within a lumen 1102, the obstruction element 1122 at least partially obstructs or occludes flow through the lumen 1102. The intraluminal anchor 1124 may also provide some obstruction to flow through the lumen 1102. In some embodiments, the intraluminal anchor 1124 may take on a variety of different shapes, some potential examples of which may be depicted in FIGS. 27A-27C. The anchor 1124 may, in some embodiments, be provided in the form of an IVC (Inferior Vena Cava) filter. The obstruction element 1122 may take any of the forms as described above in connection with obstruction element 722.

Yet another embodiment of an occlusion apparatus is described in connection with FIG. 33. The occlusion apparatus depicted in FIG. 33 includes an intraluminal anchor 1224 to which an obstruction element 1222 is attached. When deployed within a lumen 1202, the obstruction element 1222 at least partially obstructs or occludes flow through the lumen 1202. The intraluminal anchor 1224 may also provide some obstruction to flow through the lumen 1202. In some embodiments, the intraluminal anchor 1224 may take the form of a barbed SEMS (Self Expanding Metal Stent) device with the barbs interacting with the interior surface of the lumen 1202 to hold the occlusion apparatus in a selected location within the lumen 1202. The obstruction element 1222 may take any of the forms as described above in connection with obstruction element 722.

Still another embodiment of an occlusion apparatus is depicted in connection with FIG. 34 where the occlusion apparatus includes an intraluminal anchor 1324 is attached to an obstruction element 1322 through a connector 1320 that bridges a valve 1304 located within the lumen 1302. The anchor 1324 is preferably capable of holding the occlusion apparatus in a selected location within the lumen 1302. The obstruction element 1322 may take any of the forms as described above in connection with obstruction element 722.

Still another embodiment of an occlusion apparatus that may be used to occlude a lumen is depicted in FIG. 35. The occlusion apparatus depicted in FIG. 35 is in the form of a series of coils 1410 that, when expanded, can receive the lumen 1402 therein. Releasing the coils 1410 from the expanded state cause them to collapse and occlude the lumen 1402 because the coils 1410 are biased into a configuration that causes them to compress or occlude the lumen 1402.

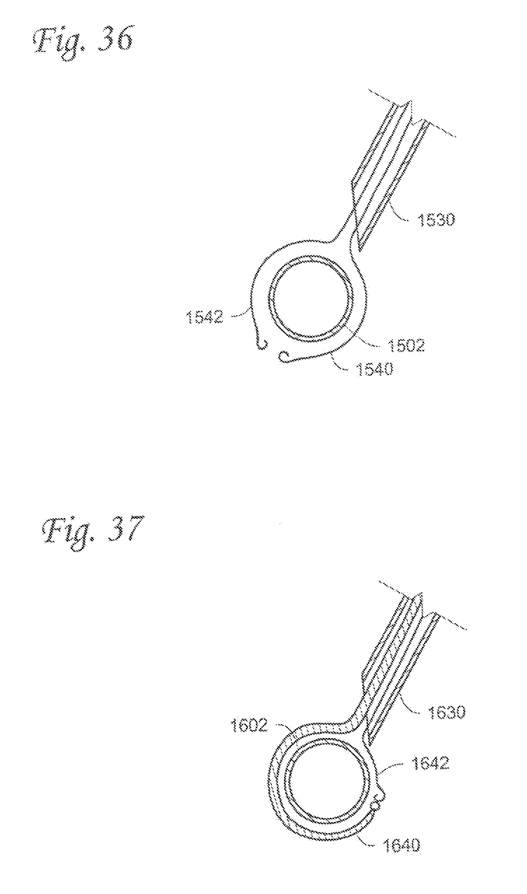

Yet another embodiment of an occlusion apparatus as described herein is depicted in FIG. 36 an includes a single or dual lumen delivery device 1530 (e.g., needle, trocar, etc.) that allows for the passage of two sutures/wires 1540 and 1542 that can connect with each other on an opposite side of a lumen 1502 such that the delivery device 1530 and the sutures/wires 1540 and 1542 can compress the lumen 1502. The connection between the sutures/wires 1540 and 1542 may be made using any suitable structure, e.g., a pair of hooks, a hook and a loop, etc.

Yet another embodiment of an occlusion apparatus as described herein is depicted in FIG. 37 an includes a single or dual lumen delivery device 1630 (e.g., needle, trocar, etc.) that allows for the passage of a J-shaped element 1640 and a suture/wire 1642 that can connect with each other such that the delivery device 1630, the J-shaped element 1640, and the suture/wire 1642 can compress the lumen 1602. The connection between the J-shaped element 1640 and the suture/wire 1642 may be made using any suitable structure, e.g., a pair of hooks, a hook and a loop, etc.

Still another embodiment of an occlusion apparatus as described herein is depicted in connection with FIGS. 38A-38D. The occlusion apparatus includes a delivery device 1730 that is located within an outer sheath 1740. The outer sheath 1740 may preferably have a piercing tip 1742 capable of piercing body tissue (e.g., lumen walls, etc.). The delivery device 1730 includes compartments 1732, one of which includes a first occlusion element 1710 and the other of which includes a second occlusion element 1710. A connector 1720 extends between the occlusion elements 1710. In the depicted embodiment, the connector 1720 is in the form of a flexible element (e.g., suture material, wire, etc.). The delivery device 1730 may include channels 1733 (see FIG. 38B) that can be used to restrain the connector 1720 such that it is restricted from twisting, etc. during the delivery process.

In some embodiments, one or both of the compartments 1732 of the delivery device 1730 preferably includes an optional ejection structure in the form of an ejector element 1734 that is capable of applying a force to the occlusion element 1710 to force it out of the compartment 1732. The ejector element 1734 may take the form of, e.g., a resilient element such as a spring, elastomeric plug, etc. In other embodiments, the ejection structure may include an expandable element (e.g., a shape memory material structure that expands when heated). In still other embodiments, the ejection structure may take the form of a balloon, bladder, or other inflatable structure.

In use, the sheath 1740 may be used to pierce tissue. With the tip 1742 of the sheath 1740 in a selected location, the sheath 1740 and the delivery device 1730 are moved relative to each other along the longitudinal axis 1701 such that the occlusion elements 1710 and the compartments 1732 in which they are located are located outside of the sheath 1740 such that the occlusion elements 1710 are forced out of the compartments 1732 (see. e.g., FIG. 38D).

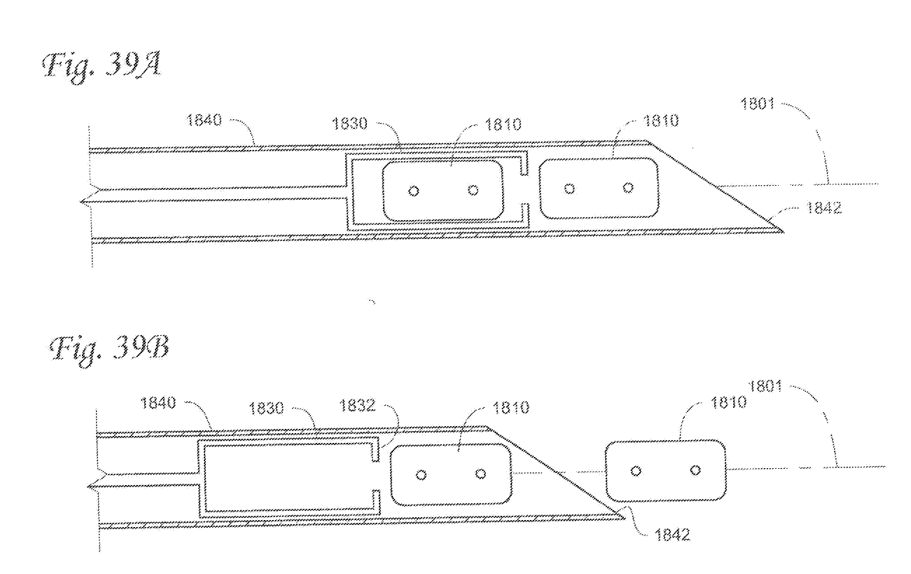

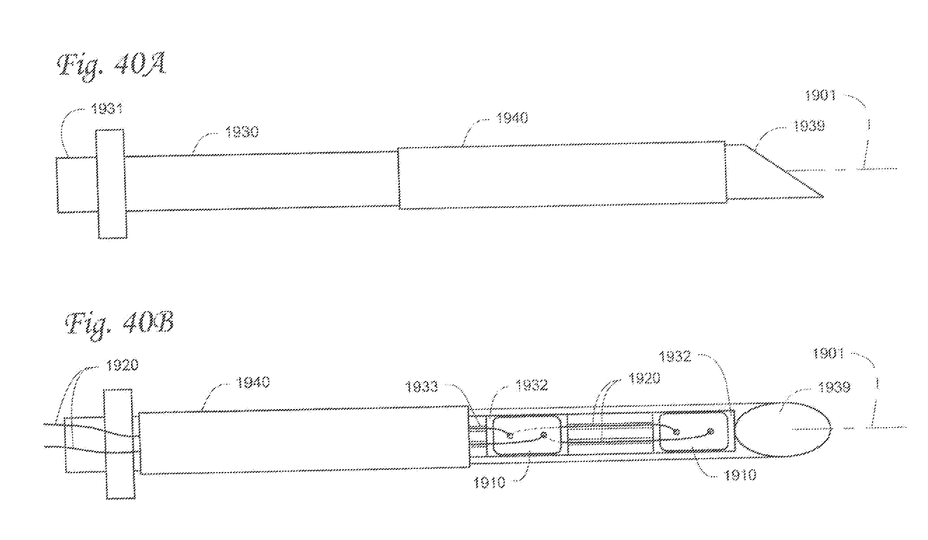

Still another embodiment of an occlusion apparatus is depicted in connection with FIGS. 39A and 39B. The depicted occlusion apparatus includes a delivery device 1830 that is located within an outer sheath 1840. The outer sheath 1840 may preferably have a piercing tip 1842 capable of piercing body tissue (e.g., lumen walls, etc.). The delivery device 1830 includes a flanged end 1832 that that can force the distal occlusion element 1810 (i.e., the occlusion element closest to the tip 1842 of the sheath 1840). A second occlusion element 1810 is located proximally from the distal occlusion element 1810. A connector (not shown) may extend between the occlusion elements 1810 in a manner similar to the other embodiments described herein.

The flanged end 1832 of the delivery device 1830 is preferably shaped such that in one rotational orientation around the longitudinal axis 1801, the distal end 1832 is positioned and shaped to push the distal occlusion element 1810 out of the lumen of the sheath 1840. Rotation of the delivery device 1830 about the axis 1801 allows it to be withdrawn proximally from the second occlusion element 1810 after deployment of the distal occlusion element 1810 (see FIG. 39B). In that position, the distal end 1832 of the delivery device 1830 can then be used to push the second, proximal occlusion element 1810 out of the sheath 1840.

Yet another embodiment of an occlusion apparatus is depicted in connection with FIGS. 40A-40D. The occlusion apparatus includes a delivery device 1930 having a movable sheath 1940 located over a portion of the delivery device 1930 that carries the occlusion elements 1910. FIG. 40A depicts the sheath 1940 in place over the occlusion elements 1910, while FIG. 40B depicts the sheath 1940 moved proximally along longitudinal axis 1901 to expose the occlusion elements 19.10 carried on the delivery device 1930.

The delivery device 1930 in the embodiment depicted in FIGS. 40A-40D includes a distal tip 1939 that is designed to pierce tissue such that the delivery device can be advanced through tissue as discussed herein.

The delivery device 1930 includes compartments 1932, one of which includes a first occlusion element 1910 and the other of which includes a second occlusion element 1910. A connector 1920 extends between the occlusion elements 1910. In the depicted embodiment, the connector 1920 is in the form of a flexible element (e.g., suture material, wire, etc.). The delivery device 1930 may include channels 1933 (see FIG. 38B) that can be used to restrain the connector 1920 such that it is restricted from twisting, etc. during the delivery process.

In some embodiments, the delivery device 1930 may include optional ejection structure in the form of one or more lumens (not shown) that lead from the proximal end 1931 to each of the compartments 1932. The lumens can be used to provide fluid pressure that, in turn, forces the occlusion elements 1910 out of the compartments 1932. In some embodiments the lumens may terminate in the compartments 1932 with one or more open orifices such that the fluid forced through the lumen exits the lumen in the compartment 1932. In some other potential embodiments, the lumens may lead to an inflatable element (e.g., a balloon, etc.) that is expanded by the fluid to force the occlusion elements 1910 out of the compartments 1932.

In use, the delivery device 1930 may be used to pierce tissue. With the tip 1939 of the delivery device 1930 in a selected location, the sheath 1940 and the delivery device 1930 are moved relative to each other along the longitudinal axis 1901 such that the occlusion elements 1910 and the compartments 1932 in which they are located are located outside of the sheath 1940 such that the occlusion elements 1910 can be forced out of the compartments 1932 (see. e.g., FIGS. 40C and 40D).

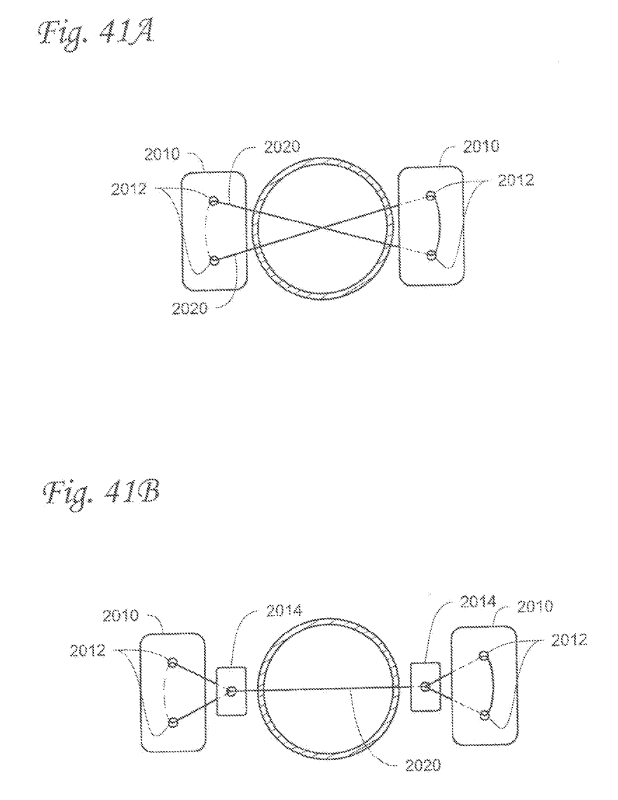

FIGS. 41A and 41B depict another optional feature that may be included in the occlusion elements as described herein. Where, for example, the occlusion elements 2010 are connected by a connector 2020 in the form of, e.g., a suture or other flexible filament-like structure that is threaded between two holes 2012 in the occlusion elements 2010, the spacing between the holes 2012 may preferably be limited to reduce potential tissue damage caused by tension between the two lengths of connector material. For example, it may be preferred that the spacing between holes 2012 in an occlusion element 2010 be about 1 millimeter or less, or potentially 0.5 millimeters or less.

To address the issue of tissue tearing, some occlusion elements may be provided with guides 2014 as depicted in FIG. 41B. The guides 2014 are preferably located between the occlusion elements 2010 to hold the lengths of connector 2020 closer together and thereby potentially reduce tension and any concomitant tearing of tissue.

Any of the occlusion apparatus described herein may be used to occlude, obstruct or otherwise close lumens alone and/or in combination energy delivery (e.g., RF tissue ablation, cryotherapy, laser, steam, sclerotherapy, etc.).

In general, the occlusion apparatus that involve compression of a lumen as described herein may be used to occlude or close a body lumen by compressing the opposite sides of the lumen together. The occlusion apparatus that include intraluminal obstruction elements may be used to occlude or close a body lumen by placing one or more obstruction elements in the body lumen. Among the body lumens that may be closed or occluded using the apparatus and/or methods described herein include, but are not limited to: blood vessels (e.g., varicose veins, etc.), uterine tubes, seminal vesicles, perforators and bleeding varicosities, fallopian tubes, male sterilization, arteriovenous malformations, etc.

In some embodiments, a delivery device in the form of a hollow needle with a stylet sealing off the distal end is introduced into the patient with, e.g., ultrasound or other guidance potentially being used to assist in proper placement of the needle. If the body lumen to be occluded is a vein, the vein may be occluded at an upstream location (using, e.g., external pressure, etc.) to reduce bleeding. The needle is then advanced through the opposing sides of the vein.

The stylet may then be removed and an occlusion element with its corresponding connector may be loaded into the needle. This may be accomplished, in some methods using a cartridge as described herein.

The first occlusion element can then be advanced through the needle using a pusher as described herein. After the first occlusion element exits the distal end of the needle the pusher is withdrawn from the delivery lumen and a second occlusion element is inserted into the delivery lumen.

The pusher is then reintroduced into the delivery lumen and the second occlusion element is advanced most of the way down the delivery lumen. The needle is then withdrawn proximally until the distal end of the needle is located proximal of the vein (i.e., the distal tip of the needle is removed from the vein).

The pusher is then used to advance the second occlusion element out of the needle such that it is located on the opposite side of the vein from the first occlusion element.

The pusher can then be withdrawn proximally from the needle and a knot (or other fixation element) is formed in the suture material and a pusher is used to advance the knot through the needle.

The two occlusion elements are then drawn together to compress the vein by using the pusher and pulling on the free ends of the suture. Additional knots and/or other fixation techniques may then be used to further secure the occlusion elements on the compressed vein. The free ends of the suture may then be severed proximate the occlusion elements and the needle can be withdrawn from the patient.

The various components of the apparatus described herein may be manufactured of any suitable material or combination of materials. Some or all of the materials may be dissolvable (e.g., bio-resorbable) materials that degrade over time without further intervention. Examples of some potentially useful materials include, but are not limited to metals, polymers, etc.

The complete disclosure of the patents, patent documents, and publications cited herein are incorporated by reference in their entirety as if each were individually incorporated.

Illustrative embodiments are discussed and reference has been made to possible variations within the scope of this invention. These and other variations and modifications will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A body lumen occlusion apparatus comprising:
a hollow delivery needle having a straight distal end and a distal outlet opening, the needle being adapted to pass transversely of a tubular body from a proximal location externally of the tubular body, through the opposing walls of the tubular body and the body lumen between the walls, to place the distal outlet opening distally of the tubular body;
a distal occlusion element contained within the needle;
a proximal occlusion element contained within the needle proximally of the distal occlusion element;
a filament-like, flexible connector disposed in the needle lumen, the connector being attached to the distal occlusion element and extending through an aperture of the proximal occlusion element, the proximal occlusion element being slidable along the connector;
whereby after the needle has been advanced through the tubular body, the distal occlusion element can be advanced out of the needle and positioned distally of the tubular body, the needle then may be retracted to position the needle outlet proximally of the tubular body and the proximal occlusion element can be advanced out of the needle and positioned proximally of the tubular body;
a pushing member advanceable through the needle lumen and engageable with the proximal occlusion member whereby, after the occlusion members have been positioned proximally and distally of the body lumen, the pushing member and flexible connector can be drawn together to cause the occlusion members to compress the walls of the tubular body together to occlude the lumen of the body member.

2. The apparatus as defined in claim 1 further comprising:
a delivery device movably disposed in the needle and associated with the occlusion elements and in engagement with the proximal occlusion element for advancing the occlusion elements toward and out of the distal outlet.

3. The apparatus as defined in claim 1 wherein the delivery device further comprises:
a first compartment and a second compartment separated from the first compartment by a barrier, wherein the first occlusion element is located in the first compartment and the second occlusion element is located in the second compartment;

4. The apparatus as defined in claim 3 further comprising ejection structure associated with the first compartment, wherein the ejection structure is configured to selectively force the first occlusion element out of the first compartment when the first compartment and the first occlusion element are outside of the delivery lumen.

5. The apparatus as defined in claim 3 further comprising ejection structure associated with the second compartment, wherein the ejection structure is configured to selectively force the second occlusion element out of the second compartment when the second compartment and the second occlusion element are outside of the delivery lumen.

6. An apparatus according to claim 4, wherein the ejection structure comprises an ejection element configured to apply a force to the first occlusion element.

7. An apparatus according to claim 6, wherein the ejection element comprises a resilient element.

8. An apparatus according to claim 6, wherein the ejection element comprises an expandable element.

9. An apparatus according to claim 8, wherein the expandable element comprises an inflatable element.

10. An apparatus according to claim 3, wherein the ejection structure comprises a lumen comprising an orifice located in the first compartment.

11. An apparatus according to claim 3, wherein the distal end of the outer sheath comprises tissue piercing structure.

12. An apparatus according to claim 3, wherein the connector extends proximally from the second occlusion element to a proximal end of the delivery device.

13. An apparatus according to claim 3, wherein the connector comprises suture material threaded through an opening in the first occlusion element and/or the second occlusion element.

14. An apparatus according to claim 13, wherein the connector comprises two lengths of suture material extending between the first occlusion element and the second occlusion element, and further wherein the delivery device comprises first and second channels extending between the first compartment and the second compartment, wherein one length of the suture material is located in the first channel and the other length of suture material is located in the second channel.

15. An apparatus according to claim 3, wherein an intraluminal obstruction element is attached to the connector and located between the first occlusion element and the second occlusion element.

16. An apparatus according to claim 13, wherein the intraluminal obstruction element comprises an embolization coil.

17. A method of using the apparatus of claim 3 to occlude a body lumen, the method comprising:
advancing the delivery device to a selected location, wherein the advancing comprises piercing two opposing sides of a body lumen;
deploying the first occlusion element on a distal side of the body lumen;
deploying the second occlusion element on the proximal side of the body lumen; and
drawing the first occlusion element and the second occlusion element together, wherein the body lumen is occluded between the first occlusion element and the second occlusion element, and wherein the first occlusion element and the second occlusion element are connected to each other through the body lumen by the connector.
